(19) United States
(12) Patent Application Publication
(10) Pub. No.: US 2018/0333070 A1
(43) Pub. Date: Nov. 22, 2018

(54) Apparatuses for Detecting and/or Destroying Abnormal Tissue, Such as Cancer Tissue, and Related Methods

(71) Applicant: NEUROFRONT TECHNOLOGY LLC, Temple, TX (US)

(72) Inventors: Samantha DAYAWANSA, Temple, TX (US); Jason HUANG, Temple, TX (US); Ekokobe FONKEM, Temple, TX (US)

(21) Appl. No.: 15/777,476

(22) PCT Filed: Nov. 18, 2016
(86) PCT No.: PCT/US16/62905

§ 371 Date: May 18, 2018

Related U.S. Application Data
(60) Provisional application No. 62/256,977, filed on Nov. 18, 2015.

(51) Int. Cl. A61B 5/05; A61B 5/00; G01R 33/04

(52) U.S. Cl. CPC A61B 5/0522; A61B 2562/0223; G01R 33/04; A61B 5/4064

(57) ABSTRACT

This disclosure includes apparatuses for detecting and/or destroying abnormal tissue, such as cancer tissue, and related methods. Some apparatuses include a device having an elongated body extending between a proximal end and a distal end, one or more detectors coupled to the distal end, each having a magnet configured to produce a magnetic field, and a magnetometer spaced apart from the magnet and configured to capture data indicative of a magnetic flux density. In some apparatuses, the magnet of a detector is configured to produce a constant magnetic field. In some apparatuses, the distal end of the elongated body is configured to be inserted into a target site on a patient.

This application claims the benefit of priority to U.S. Provisional Patent Application Ser. No. 62/256,977, filed Nov. 18, 2015, hereby incorporated by reference in its entirety.

### BACKGROUND

### 1. Field of Invention

The present invention relates generally to oncology, and more specifically, but not by way of limitation, to apparatuses for detecting and/or destroying abnormal tissue, such as cancer tissue, and related methods.

### 2. Description of Related Art

Abnormal tissue, such as cancer tissue, may be treated in a number of ways, such as, for example, through surgery, radiation, chemotherapy, and/or a combination thereof. Due in part to having a relatively high recurrence rate, certain types of cancer tissue may be associated with a relatively low survival rate, even when treated.

One example of such cancer tissue may be a brain tumor or glioblastoma. Often times, a glioblastoma is treated through a combination of a debulking surgery (e.g., to remove a portion of the glioblastoma, thereby relieving pressure on surrounding tissue) and/or radiation and/or chemotherapy in an attempt to remove the remainder of the glioblastoma. Unfortunately, in many instances, portions of the glioblastoma that are not removed during the debulking surgery may cause recurrence of the glioblastoma.

Examples of cancer detection apparatuses are disclosed in Pub. No. US 2012/0035457.

### SUMMARY

Some embodiments of the present apparatuses for in vivo detection of abnormal tissue comprise: a device including an elongated body extending between a proximal end and a distal end, one or more detectors coupled to the distal end, each having a magnet and a magnetometer spaced apart from the magnet and configured to capture data indicative of a magnetic flux density.

In some embodiments, the one or more detectors comprises a plurality of detectors. In some embodiments, each of the one or more detectors is movable between a deployed state and a retracted state in which the magnet is closer to the magnetometer than when the detector is in the deployed state. Some embodiments comprise one or more proximity or contact sensors, each coupled to the distal end of the elongated body.

In some embodiments, the magnet of each of the one or more detectors is configured to produce a constant magnetic field. In some embodiments, the magnet of each of the one or more detectors is configured to produce a varying magnetic field. In some embodiments, the magnet of each of the one or more detectors comprises a permanent magnet. In some embodiments, the magnet of each of the one or more detectors comprises an electromagnet.

In some embodiments, the magnetometer of each of the one or more detectors comprises a fluxgate magnetometer. In some embodiments, the magnet of each of the one or more detectors is spaced apart from the magnetometer of the detector by a distance of approximately 0.5 mm.

In some embodiments, the device comprises, for each of the one or more detectors, one or more wires configured to be in electrical communication with the magnet. In some embodiments, the device comprises, for each of the one or more detectors, one or more wires configured to be in electrical communication with the magnetometer. In some embodiments, at least one of the one or more wires is at least partially disposed within the body. In some embodiments, at least one of the one or more wires comprises a substantially non-ferromagnetic material. In some embodiments, the substantially non-ferromagnetic material comprises at least one of: silver, copper, gold, and aluminum.

In some embodiments, each of the one or more detectors comprises a plate coupled to the magnet on a side of the magnet that is opposite the magnetometer. In some embodiments, each of the one or more detectors comprises a plate coupled to the magnetometer on a side of the magnetometer that is opposite the magnet.

In some embodiments, the distal end of the body defines a flange such that at least a portion of the flange is proximal to the one or more detectors. In some embodiments, the flange is movable between a collapsed state, in which the flange has a first maximum transverse dimension, and a deployed state, in which the flange has a second maximum transverse dimension that is larger than the first maximum transverse dimension. In some embodiments, movement of the flange toward the deployed state causes movement of each of the one or more detectors toward the deployed state.

In some embodiments, the body includes a pivotal connection between the distal end and the proximal end, the pivotal connection configured to permit angular displacement of the distal end relative to the proximal end. In some embodiments, the device includes one or more rods or one or more wires configured to actuate the pivotal connection to angularly displace the distal end relative to the proximal end. In some embodiments, the device includes one or more actuators configured to actuate the pivotal connection to angularly displace the distal end relative to the proximal end.

In some embodiments, the body is configured such that a length of the body between the proximal end and the distal end is adjustable. In some embodiments, the body comprises a plurality of telescoping segments configured to permit adjustment of the length of the body. In some embodiments, the device includes one or more rods or one or more wires configured to actuate the plurality of telescoping segments to adjust the length of the body. In some embodiments, the device includes one or more actuators configured to actuate the plurality of telescoping segments to adjust the length of the body.

In some embodiments, the body includes an interior passageway extending through the distal end. In some embodiments, the device comprises a trocar configured to be coupled to the body such that the trocar extends from the distal end. In some embodiments, the trocar is configured to be coupled to the body such that the trocar extends from the distal end of the body a distance that is between approximately 3 mm and approximately 5 mm. In some embodiments, the trocar is configured to be coupled to the body such that the trocar extends between the magnet and the magnetometer of at least one of the one or more detectors. In some embodiments, the trocar is slidably disposed within the interior passageway of the body.

In some embodiments, the trocar has a diameter of approximately 0.1 mm. In some embodiments, the trocar comprises a substantially non-ferromagnetic material. In some embodiments, the substantially non-ferromagnetic material comprises at least one of: silver, gold, copper, and aluminum.

In some embodiments, the trocar is configured to be in electrical communication with an electrical power source. Some embodiments comprise the electrical power source. In some embodiments, the trocar includes a lumen in fluid communication with the interior passageway of the body. In some embodiments, the trocar is configured to convey a cryoablative fluid from a cryoablative fluid source and through the lumen. Some embodiments comprise the cryoablative fluid source.

In some embodiments, the distal end of the body is configured to be inserted into a target site on a patient. In some embodiments, the distal end of the device is disposable through a lumen of an elongated delivery device. In some embodiments, the delivery device comprises a needle. In some embodiments, the delivery device comprises a steerable delivery device. In some embodiments, a minimum transverse dimension of the lumen of the delivery device is 0.9 mm or smaller. Some embodiments comprise the delivery device.

Some embodiments comprise one or more processors configured to detect a presence of abnormal tissue based, at least in part, on data captured by the magnetometer of at least one of the one or more detectors. In some embodiments, the one or more processors are configured to detect a presence of abnormal tissue at least by comparing data captured by the magnetometer of at least one of the one or more detectors to a baseline magnetic flux density.

In some embodiments, the device comprises a receiver in communication with at least one of the one or more actuators, the receiver configured to receive one or more commands indicative of at least one of a desired angular position of the distal end of the body relative to the proximal end and a desired length of the body and communicate the one or more commands to at least one of the one or more actuators. In some embodiments, the device comprises a transmitter in communication with at least one of the one or more detectors and configured to transmit data captured by the magnetometer of the detector to the one or more processors. Some embodiments comprise a display configured to be coupled to the one or more processors and to display an image indicative of data captured by the magnetometer of at least one of the one or more detectors.

Some embodiments of the present methods comprise detecting a presence of abnormal tissue using any apparatus and/or device of the present disclosure. Some embodiments of the present methods comprise: inserting a detector of a device into a target site on a patient, the detector including a magnet and a magnetometer, producing a magnetic field with the magnet, moving the detector relative to the patient, and capturing, with the magnetometer, data indicative of disturbances in the magnetic field. In some embodiments, the target site is located within the patient's brain.

In some embodiments, the inserting comprises forming a cavity at or proximate to the target site and inserting the detector into the cavity. In some embodiments, forming the cavity comprises cryoablation. In some embodiments, forming the cavity comprises electrocauterization. In some embodiments, the inserting comprises inserting an elongated delivery device into the target site, the detector being disposed within a lumen of the delivery device, removing the detector from the lumen of the delivery device, and removing the delivery device from the target site.

Some embodiments comprise analyzing data captured by the magnetometer to detect a presence of abnormal tissue at or proximate to the target site. In some embodiments, data captured by the magnetometer includes data indicative of a magnetic flux density. In some embodiments, the analyzing comprises comparing data captured by the magnetometer to a baseline magnetic flux density.

In some embodiments, the moving comprises moving the detector along a boundary of the cavity. Some embodiments comprise ablating tissue at or proximate to the target site. In some embodiments, the ablating comprises cryoablation. In some embodiments, the ablating comprises electrocauterization.

Some embodiments of the present apparatuses for in vivo detection of abnormal tissue comprise: a device including a detector configured to be inserted into a patient, the detector having a magnet configured to produce a magnetic field and a magnetometer spaced apart from the magnet and configured to capture data indicative of a magnetic flux density, and a processor configured to analyze data captured by the magnetometer to detect a presence of abnormal tissue within the patient. In some embodiments, the detector is disposable through a lumen of an elongated delivery device. In some embodiments, the processor is configured to analyze data captured by the magnetometer at least by comparing data captured by the magnetometer to a baseline magnetic flux density.

In some embodiments, the device comprises one or more flexible wires, at least one of the one or more flexible wires is configured to be in electrical communication with the magnetometer, and, optionally, at least one of the one or more flexible wires is configured to be in electrical communication with the magnet.

In some embodiments, the device includes an elongated body extending between a proximal end and a distal end, and the detector is coupled to the distal end of the elongated body. In some embodiments, the detector is movable between a deployed state and a retracted state in which the magnet is closer to the magnetometer than when the detector is in the deployed state. In some embodiments, the distal end of the elongated body defines a flange such that at least a portion of the flange is proximal to the detector, and the flange is movable between a collapsed state in which the flange has a first maximum transverse dimension and a deployed state in which the flange has a second maximum transverse dimension that is larger than the first maximum transverse dimension. In some embodiments, movement of the flange toward the deployed state causes movement of the detector toward the deployed state.

Some embodiments of the present methods comprise: inserting a detector of a device into a target site of a patient, the detector including a magnet and a magnetometer, producing a magnetic field with the magnet, and capturing, with the magnetometer, data indicative of a magnetic flux density and/or disturbances in the magnetic field. In some embodiments, the inserting the detector comprises inserting an elongated delivery device into the target site, the detector being disposed within a lumen of the delivery device, removing the detector from the lumen of the delivery device, and removing the delivery device from the target site.

In some embodiments, the device comprises one or more flexible wires, at least one of the one or more flexible wires is configured to be in electrical communication with the magnetometer, and, optionally, at least one of the one or more flexible wires is configured to be in electrical communication with the magnet. In some embodiments, the device includes an elongated body extending between a proximal end and a distal end, and the detector is coupled to the distal end of the elongated body.

In some embodiments, the capturing data with the magnetometer is performed while producing the magnetic field with the magnet. In some embodiments, the magnetic field produced by the magnet is constant. In some embodiments, the capturing data with the magnetometer is performed before producing the magnetic field with the magnet. In some embodiments, the producing the magnetic field is performed in response to data captured by the magnetometer (e.g., when data captured by the magnetometer is indicative of the presence of abnormal tissue). Some embodiments comprise increasing an intensity of the magnetic field produced by the magnet in response to data captured by the magnetometer.

Some embodiments comprise analyzing data captured by the magnetometer to detect a presence of abnormal tissue at or proximate to the target site. In some embodiments, the analyzing data captured by the magnetometer comprises comparing data captured by the magnetometer to a baseline magnetic flux density.

In some embodiments, the target site is within the patient's brain. In some embodiments, the target site of the patient comprises a cavity that was previously occupied by abnormal tissue. Some embodiments comprise moving the detector along a boundary of the cavity. In some embodiments, the patient has been previously diagnosed as having cancer and/or the patient has been determined to have a genetic predisposition to cancer.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, an apparatus that “comprises,” “has,” “includes,” or “contains” one or more elements possesses those one or more elements, but is not limited to possessing only those elements. Likewise, a method that “comprises,” “has,” “includes,” or “contains” one or more steps possesses those one or more steps, but is not limited to possessing only those one or more steps.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 depicts one embodiment of the present apparatuses.

FIGS. 2A-2C are perspective, cross-sectional side, and front views, respectively, of a detector, which may be suitable for use in some embodiments of the present apparatuses.

FIGS. 3A and 3B illustrate steps of one embodiment of the present methods for detecting abnormal tissue.

FIGS. 4A and 4B are cross-sectional side views of a first embodiment of a device, which may be suitable for use in some embodiments of the present apparatuses, shown in a first position and a second position, respectively.

FIG. 5 is a front view of a second embodiment of a device, which may be suitable for use in some embodiments of the present apparatuses.

FIG. 6 is a side view of a third embodiment of a device, which may be suitable for use in some embodiments of the present apparatuses.

### DETAILED DESCRIPTION OF ILLUSTRATIVE EMBODIMENTS

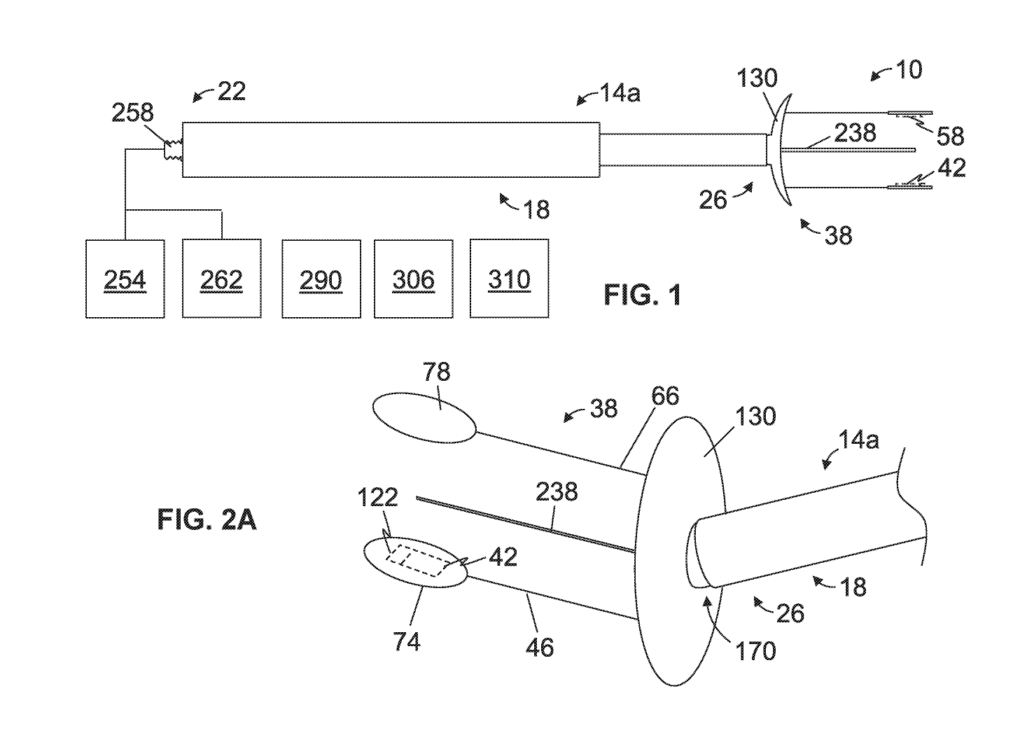

Referring now to the figures, and more particularly to FIG. 1, shown therein and designated by the reference numeral 10 is one embodiment of the present apparatuses. In the embodiment shown, apparatus 10 includes a device 14a having an elongated body 18 (e.g., which may comprise multiple components, such as, for example, flange 130, component(s) for pivotal connection 170 (e.g., ball 174 and socket 178), telescoping segments 202a and 202b, and/or the like) extending between a proximal end 22 and a distal end 26. In this embodiment, device 14a is configured to identify or detect abnormal tissue, such as, for example, cancer tissue. More specifically, in the depicted embodiment, device 14a includes one or more detectors 38 coupled to distal end 26 of body 18, each configured to detect abnormal tissue.

As described in more detail below, when subjected to a magnetic field, abnormal tissue, such as cancer tissue, may have a measurably different effect on the magnetic field than normal tissue. Thus, in the embodiment shown, device 14a may be configured to detect abnormal tissue, such as cancer tissue, by capturing data indicative of magnetic field characteristic(s) while the abnormal tissue is subjected to the magnetic field.

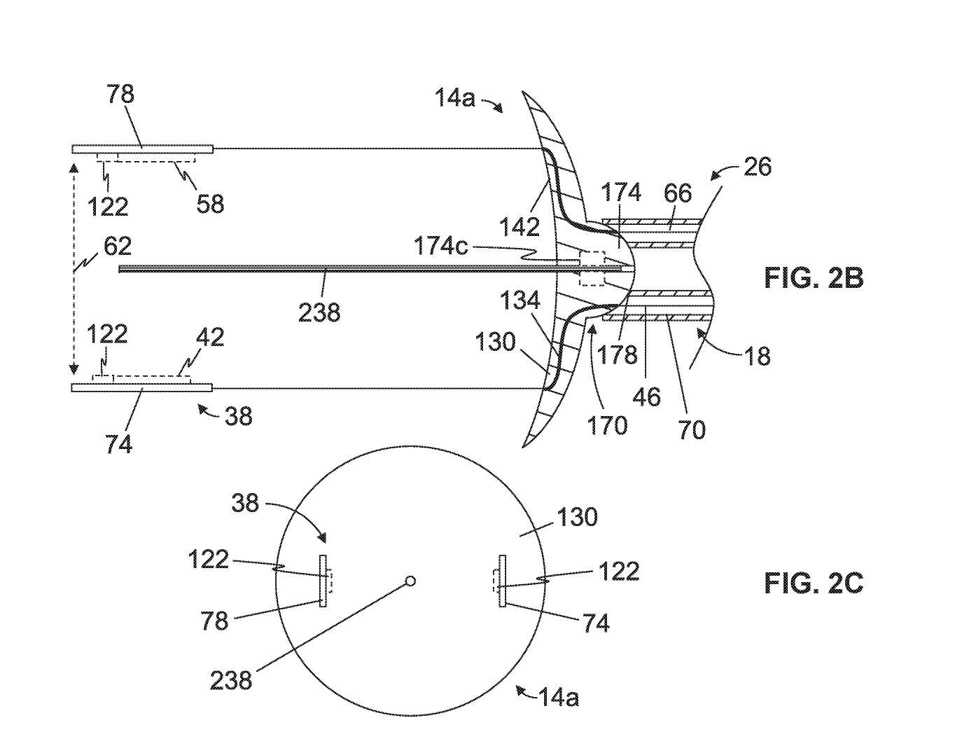

Referring additionally to FIGS. 2A-2C, provided by way of example, in this embodiment, each detector 38 includes a magnet 42, and more particularly, an electromagnet, configured to produce a magnetic field, whether constant and/or varying. Magnet(s) (e.g., 42) configured to produce a constant magnetic field may mitigate the formation of eddy currents within tissue that create secondary magnetic fields, which, in some instances, may complicate detection of abnormal tissue (e.g., by disturbing the primary magnetic field); however, in other instances, magnet(s) (e.g., 42) configured to produce a varying magnetic field may provide for more accurate detection of abnormal tissue. Electrical power to such magnet(s) (e.g., 42) may be provided by a power source, such as, for example, a battery (coupled to or disposed within a device 14a, 14b, 14c, and/or the like), a power source that is external to, but is in electrical communication with, the device, and/or the like. Nevertheless, in other embodiments, a detector (e.g., 38) may comprise any suitable magnet, such as, for example, a permanent magnet, which may comprise any suitable (e.g., ferromagnetic) material, such as, for example, neodymium iron boron, samarium cobalt, strontium ferrite, aluminum nickel cobalt, and/or the like.

In the depicted embodiment, each detector 38 includes a magnetometer 58 spaced apart from magnet 42 and configured to capture data indicative of characteristic(s) of a magnetic field, such as, for example, a flux density of the magnetic field at or proximate to the magnetometer. In the embodiment shown, for each detector 38, magnetometer 58 is spaced apart from magnet 42 by a distance 62 (e.g., when the detector is in a deployed state, as described in more detail below). In this embodiment, distance 62 is approximately 0.5 mm; however, in other embodiments, a distance (e.g., 62) between a magnet (e.g., 42) and a magnetometer (e.g., 58) may be any suitable distance, such as, for example, less than 0.5 mm or greater than 0.5 mm (e.g., 0.6, 0.7, 0.8, 0.9, 1.0, 1.5, 2.0, 2.5, 3.0, 3.5, 4.0, 4.5, 5 mm, or larger). Magnetometers (e.g., 58) of the present devices may comprise any suitable magnetometer, such as, for, example, a fluxgate magnetometer, and/or the like. Examples of suitable magnetometers (e.g., 58) are provided in: (1) Jian Lei et al., Micro Fluxgate Sensor using Solenoid Coils Fabricated by MEMS Technology, 12 MEASUREMENT SCIENCE REVIEW 286-289 (2012), available at http://www.measurement.sk/2012/JianLei.pdf; (2) Pub. No. US 2011/0074408; (3) U.S. Pat. No. 5,644,230; (4) U.S. Pat. No. 5,762,064; and (5) U.S. Pat. No. 8,054,073, each of which is hereby incorporated by reference in its entirety.

In the embodiment shown, each magnet 42 and/or each magnetometer 58 may have a maximum transverse dimension that is approximately 1 mm or smaller, to facilitate, for example, disposal of the magnet and/or magnetometer through a lumen 114 of a delivery device 110 (e.g., an 18 gauge lumen, having an inner diameter of 0.838 mm); however, detector(s) (e.g., 38) of other embodiments may include magnet(s) (e.g., 42) and/or magnetometer(s) (e.g., 58) of any suitable dimensions, such as, for example, magnet(s) and/or magnetometer(s) having a maximum transverse dimension that is larger than 1 mm (e.g., 1.5, 2.0, 2.5, 3.0, 3.5, 4.0, 4.5, 5 mm, or larger). Magnet(s) (e.g., 42) and/or magnetometer(s) (e.g., 58) of the present detectors (e.g., 38) may comprise any suitable shape, such as, for example, disk-, block-, ring-, arc-, and/or sphere-shaped and/or the like. Magnet(s) (e.g., 42) and/or magnetometers (e.g., 58) of the present detectors (e.g., 38) may be coated with a biocompatible material, such as, for example, a metal (e.g., a titanium alloy, and/or the like), a ceramic (e.g., aluminum oxide, zirconia, calcium phosphate, and/or the like), a polymer (e.g., a silicone, polyethylene, polyvinyl chloride, polyurethane, polyacetide, collagen, gelatin, elastin, silk, polysaccharide, and/or the like), and/or the like, which may be biodegradable.

In this embodiment, device 14a comprises, for each detector 38, one or more wires 46 configured to be in electrical communication with magnet 42 to, for example, allow for activation of the magnet, control over an intensity (e.g., strength and/or magnetic flux density) and/or direction of a magnetic field generated by the magnet, and/or the like. In the depicted embodiment, device 14a comprises, for each detector 38, one or more wires 66 configured to be in electrical communication with magnetometer 58 to, for example, allow for activation and/or control of the magnetometer, transmission of data captured by the magnetometer, and/or the like. In this embodiment, wire(s) 46 and/or 66 may be of sufficient stiffness (or may comprise portion(s) of sufficient stiffness) to support magnet 42 and/or magnetometer 58, respectively, in an operative position (e.g., the position depicted in FIG. 2A). In the embodiment shown, at least one of wire(s) 46 and/or at least one of wire(s) 66 may be at least partially disposed within body 18 (e.g., within an interior channel 70 of the body). In this embodiment, at least one of wire(s) 46 and/or at least one of wire(s) 66 comprises a substantially non-ferromagnetic material, which may mitigate interference of the wire(s) with magnetometer 58 and/or a magnetic field generated by magnet 42. Such non-ferromagnetic materials may include, for example, silver, copper, gold, aluminum, and/or the like. Wire(s) 46 and/or 66 may be coated with an insulating material, such as, for example, a rubber, a plastic, and/or the like.

In the embodiment shown, each of one or more detectors 38 includes a plate 74 coupled to magnet 42 on a side of the magnet that is opposite magnetometer 58. Similarly, in this embodiment, each of one or more detectors 38 includes a plate 78 coupled to magnetometer 58 on a side of the magnetometer that is opposite magnet 42. Such plates (e.g., 74, 78, and/or the like) of a detector (e.g., 38) may provide a mounting location and/or support for a magnet (e.g., 42) and/or magnetometer (e.g., 58) and may be ferromagnetic (e.g., to direct or focus a magnetic field generated by the magnet to a region between and extending from between the magnet and magnetometer, which may increase a sensitivity of the detector, shield objects outside of the region from the magnetic field and/or prevent those objects from interfering with the magnetic field, increase an intensity of the magnetic field, and/or the like) and/or non-ferromagnetic (e.g., to mitigate interference of the plates with the magnetometer and/or a magnetic field generated by the magnet). In this embodiment, plate 74 and/or plate 78 may comprise a biocompatible material, such as, for example, one or more of those described above, and may be biodegradable.

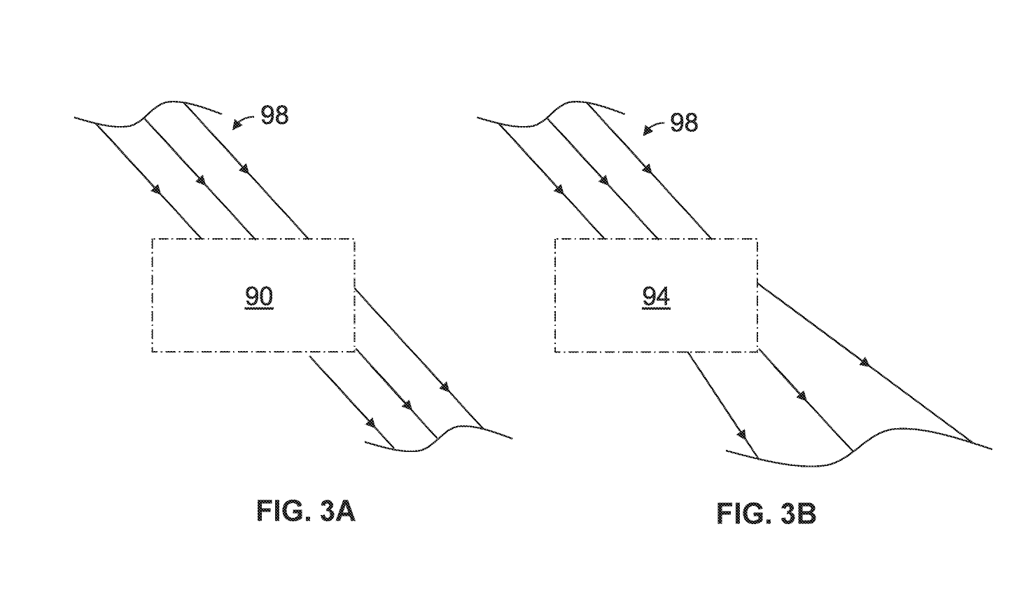

As mentioned above, when subjected to a magnetic field, abnormal tissue, such as cancer tissue, may have a measurably different effect on the magnetic field than normal tissue. For example, and referring additionally to FIGS. 3A and 3B, shown are normal issue 90 (FIG. 3A) and cancer tissue 94 (FIG. 3B) subjected to a magnetic field 98, which is represented by lines of magnetic flux. As shown, normal tissue 90 may have a relatively small effect on magnetic field 98; for example, despite the presence of normal tissue, characteristic(s), such as, for example, flux density, of the magnetic field may remain relatively unchanged. However, cancer tissue 94 may absorb and/or redirect magnetic field 98, resulting in detectable changes in characteristic(s), such as, for example flux density, of the magnetic field (e.g., at least when compared to the effect of normal tissue 90 on the magnetic field, shown in FIG. 3A).

Such detectable differences between the effects of normal tissue 90 and cancer tissue 94 on magnetic field 98 may be caused, for example, by the cancer tissue having a higher magnetic permeability than the normal tissue, cells of the cancer tissue replicating more quickly than cells of the normal tissue (e.g., causing distortions or disturbances of the magnetic field (e.g., over time), which may not be caused, at least in the same quantity or magnitude or at the same rate, by the normal tissue). Thus, in this embodiment, device 14a may be configured to detect abnormal tissue, such as cancer tissue 94, by capturing data indicative of magnetic field 98 characteristic(s) in the presence of abnormal tissue, whether directly or by comparison to characteristic(s) of the magnetic field in the presence of normal tissue 90.

Abnormal tissue, such as cancer tissue, may produce a magnetic field that differs (e.g., in intensity and/or frequency) from a magnetic field produced by normal tissue. For example, tissue growth may be detectable in the electromagnetic spectrum, and abnormal tissue may grow at a faster rate than, and thus produce a different magnetic field than, normal tissue. This difference may be particularly noticeable for certain types of abnormal and normal tissues. For example, abnormal tissue in a patient's brain (e.g., a glioblastoma) may have a relatively high growth rate when compared to normal tissue in the patient's brain, which may grow little to none. Thus, in some embodiments, a magnetometer (e.g., 58) can be used to determine whether tissue is abnormal or normal by detecting a magnetic field produced by the tissue (e.g., without applying a magnetic field to the tissue with a magnet 42).

Magnetic fields (e.g., 98) may be used to prevent and/or slow growth of and/or induce death of abnormal tissue. For discussion on this point, see Kirson, Eilon D. et al. “Alternating Electric Fields Arrest Cell Proliferation in Animal Tumor Models and Human Brain Tumors.” Proceedings of the National Academy of Sciences of the United States of America 104.24 (2007): 10152-10157. PMC. Web. 17 Nov. 2016, which is hereby incorporated by reference in its entirety. Thus, in some embodiments, abnormal tissue may be prevented from growing, have its growth rate reduced, and/or be destroyed via control of a magnet (e.g., 42) (e.g., by producing a magnetic field with the magnet, varying an intensity and/or frequency of the magnetic field, and/or the like). In some embodiments, a magnetic field can be produced by a magnet (e.g., 42), an intensity and/or frequency of the magnetic field can be varied, and/or the like in response to (e.g., upon) detection of abnormal tissue by a magnetometer (e.g., 58).

In the depicted embodiment, distal end 26 of body 18 is configured to be inserted into a target site on a patient. For example, in the embodiment shown, distal end 26 of body 18 is disposable through a lumen 114 of an elongated delivery device 110, which may facilitate insertion of the distal end into a target site on a patient. In the embodiment shown, delivery device 110 comprises a needle; however, in other embodiments, a delivery device (e.g., 110) may comprise any suitable structure that is capable of delivering a device (e.g., 14a, 14b, 14c and/or the like), or at least a distal end (e.g., 26) thereof, into a target site on a patient. For example, some embodiments may include a steerable delivery device (e.g., 110), suitable examples of which are disclosed in: (1) U.S. Pat. No. 7,918,845; (2) U.S. Pat. No. 8,920,369; and (3) Pub. No. US 2012/0024099, each of which is hereby incorporated by reference in its entirety. In such embodiments, a steerable delivery device (e.g., 110) may facilitate positioning of a device (e.g., 14a, 14b, 14c and/or the like) at or proximate to abnormal tissue within a patient, which may be aided by computerized tomography (CT), magnetic resonance imaging (MRI), and/or the like.

In this embodiment, lumen 114 of delivery device 110 has a minimum transverse dimension 118 that is 0.9 mm or smaller (e.g., the lumen is an 18 gauge lumen, having an inner diameter of 0.838 mm) (e.g., to minimize trauma to the patient during insertion of the delivery device); however, other embodiments may include a delivery device (e.g., 110) having a lumen (e.g., 114) with any suitable dimensions, such as, for example, having a minimum transverse dimension of 1, 1.5, 2.0, 2.5, 3, 3.5, 4.0, 4.5, 5 mm, or larger.

In the embodiment shown, device 14a comprises one or more proximity or contact sensors 122, each coupled to distal end 26 of body 18. Such proximity or contact sensor(s) (e.g., 112) may comprise any suitable sensor, such as, for example, a pressure-sensitive, ultrasonic, laser-based, and/or the like sensor. At least through such proximity or contact sensor(s) (e.g., 122), some embodiments of the present devices (e.g., 14a, 14b, 14c, and/or the like) may facilitate positioning of the device at or proximate to abnormal tissue within a patient.

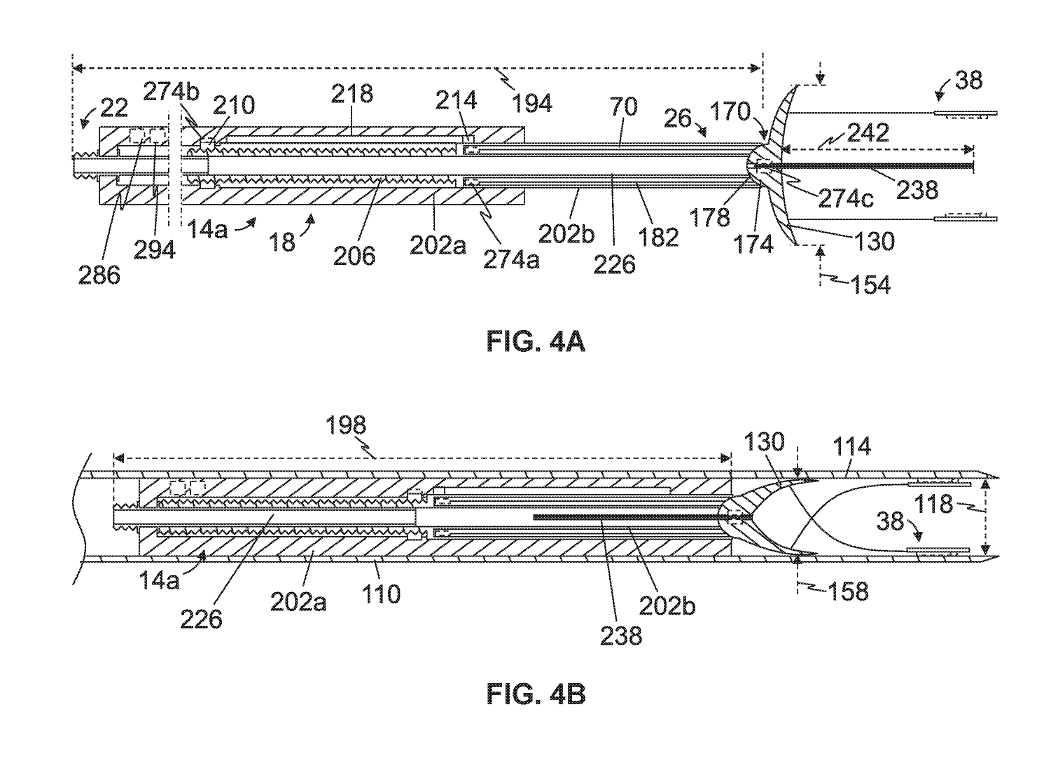

Referring additionally to FIGS. 4A and 4B, in the depicted embodiment, distal end 26 of body 18 includes a flange 130. In the embodiment shown, flange 130 is proximal to one or more detectors 38. For example, in this embodiment, flange 130, for each detector 38, defines one or more interior channels 134, each configured to receive at least one of wire(s) 46 and/or 66 coupled to magnet 42 and/or magnetometer 58, respectively. In the depicted embodiment, flange 130 comprises a circular cross-section (FIG. 2C); however, in other embodiments, a flange (e.g., 130) may comprise any suitable shape, such as, for example, a shape having a cross-section that is elliptical and/or otherwise rounded, triangular, square, and/or otherwise polygonal, and/or the like. For further example, in the embodiment shown, flange 130 has a concave distal surface 142; however, in other embodiments, a distal surface (e.g., 142) of a flange (e.g., 130) may be convex or flat.

Flanges (e.g., 130) of the present devices (e.g., 14a, 14b, and/or the like) may be ferromagnetic (e.g., to direct or focus a magnetic field generated by a magnet 42 of a detector 38 to and away from distal portions of the device, which may increase a sensitivity of the detector, shield objects and/or portions of the device proximal to the flange from the magnetic field and/or prevent those objects and/or portions from interfering with the magnetic field, increase an intensity of the magnetic field, and/or the like) and/or non-ferromagnetic (e.g., to mitigate interference of the flange with a magnetometer 58 of the detector and/or the magnetic field generated by the magnet). In the depicted embodiment, flange 130 may comprise a biocompatible material, such as, for example, one or more of those described above, and may be biodegradable.

Referring additionally to FIGS. 4A and 4B, in the embodiment shown, each detector 38 is movable between a deployed state (e.g., FIG. 4A) and a retracted state (e.g., FIG. 4B) in which, for at least this embodiment, magnet 42 is closer to magnetometer 58 than when the detector is in the deployed state. For example, in this embodiment, magnet 42 and magnetometer 58 of at least one detector 38 are both coupled to flange 130 (e.g., via wire(s) 46 and wire(s) 66, respectively). In the depicted embodiment, flange 130 is movable between a deployed state (e.g., FIG. 4A) and a collapsed state (e.g., FIG. 4B), and thus, movement of the flange towards the deployed state may cause movement of each detector 38 coupled to the flange toward the deployed state. For example, in the embodiment shown, flange 130 may be flexible such that, when device 14a is disposed within lumen 114 of delivery device 110, the flange may be moved by the delivery device to the collapsed state, and, when the device is removed from the lumen of the delivery device, the flange may move (e.g., resiliently) to the deployed state. Of course, in other embodiments, a flange (e.g., 130) of a device (e.g., 14a, 14b, and/or the like) may be movable between a deployed state and a collapsed or retracted state in any suitable fashion or may not be movable between a deployed state and a collapsed state.

In this embodiment, flange 130, when in the deployed state (e.g., FIG. 4A) has a first maximum transverse dimension 154 that is 2 mm or smaller; however, other embodiments may include a device (e.g., 14a) comprising a flange (e.g., 130) that has any suitable dimensions, such as, for example having a maximum transverse dimension (in a deployed state, if deployable) of 1, 1.5, 2.0, 2.5, 3.0, 3.5, 4.0, 4.5, 5.0 mm, or larger. In the depicted embodiment, flange 130, when in the collapsed state (e.g., FIG. 4B) has a second maximum transverse dimension 158 that is smaller than first maximum transverse dimension 154 and, in the embodiment shown, may correspond to minimum transverse dimension 118 of lumen 114 of delivery device 110.

In the embodiment shown, body 18 includes a pivotal connection 170 between distal end 26 and proximal end 22 and configured to permit angular displacement of the distal end relative to the proximal end. In this embodiment, pivotal connection 170 comprises a ball 174 movably received by a socket 178, such that, for example, distal end 26 may be angularly displaced relative to proximal end 22 in at least two degrees of freedom. However, other embodiments may include a device (e.g., 14a, 14b, and/or the like) having a body (e.g., 18) with any suitable pivotal connection (e.g., 170) between a distal end (e.g., 26) and a proximal end (e.g., 22), such as, for example, a hinge (e.g., to permit angular displacement of the distal end relative to the proximal end in at least one degree of freedom), and/or the like, or without a pivotal connection (e.g., 170).

Such a pivotal connection (e.g., 170) between a distal end (e.g., 26) and a proximal end (e.g., 22) of a body (e.g., 18) may be actuated in any suitable fashion, and the following description is provided only by way of example. In the depicted embodiment, device 14a includes one or more rods or wires 182 coupled to pivotal connection 170, and more particularly, to ball 174, and configured to actuate the pivotal connection to angularly displace distal end 26 relative to proximal end 22. For example, in the embodiment shown, movement of one of rod(s) or wire(s) 182 may rotate ball 174 within socket 178, and thus, distal end 26 relative to proximal end 22. In the embodiment shown, at least one of rod(s) or wire(s) 182 may be at least partially disposed within body 18 (e.g., within an interior channel 70 of the body). One or more rods or wires (e.g., 182) and thus a pivotal connection (e.g., 170) of a device (e.g., 14a, 14b, and/or the like) may be actuated by one or more actuators 274a (described in more detail below) and/or by physical interaction between a user and the device (e.g., via a user-operable adjustment member, such as, for example, a knob, slider, joystick, and/or the like, which may be coupled to the device).

In this embodiment, body 18 is configured such that a length of the body between proximal end 22 and distal end 26 is adjustable. For example, in the depicted embodiment, body 18 is movable between an extended position (e.g., FIG. 4A), in which the body has a first length 194 between proximal end 22 and flange 130 of distal end 26, and a retracted position (e.g., FIG. 4B), in which the body has a second length 198 between the proximal end and the flange of the distal end that is smaller than the first length. In some embodiments, a first length (e.g., 194) may be greater than any one of, or between any two of: 5, 6, 7, 8, 9, 10, 11, 12, 13, 14, 15, 16, 17, 18, 19, or 20 centimeters (cm) and a second length (e.g., 198) may be greater than any one of, or between any two of: 4, 5, 6, 7, 8, 9, or 10 cm. In the embodiment shown, body 18 includes a plurality of telescoping segments (e.g., 202a and 202b, in this embodiment) that permit adjustment to a length of the body between proximal end 22 and distal end 26; however, in other embodiments, such adjustability may be accomplished in any suitable fashion or may not be present. Telescoping segments (e.g., 202a and 202b) are exemplary, as the present devices (e.g., 14a, 14b, and/or the like) may include bodies (e.g., 18) having any suitable telescoping structures, such as, for example, those described in: (1) U.S. Pat. No. 8,298,188; and (2) U.S. Pat. No. 5,882,344, each of which is hereby incorporated by reference in its entirety.

Telescoping segments (e.g., 202a, 202b, and/or the like) of a body (e.g., 18) of a device (e.g., 14a, 14b, and/or the like) may be actuated in any suitable fashion, and the following description is provided only by way of example. In this embodiment, device 14a includes a threaded shaft 206 threadably received by a threaded driving member or nut 210, such that, for example, rotation of the driving member or nut relative to the threaded shaft causes translation of the threaded shaft relative to the driving member or nut. In the depicted embodiment, driving member or nut 210 is coupled to one of the telescoping segments (e.g., 202a) and threaded shaft 206 is coupled to one other of the telescoping segments (e.g., 202b) such that translation of the threaded shaft relative to the driving member or nut causes translation of the one of the telescoping segments relative to the one other of the telescoping segments (and thus, adjustment of a length of body 18). In the embodiment shown, one of the telescoping segments (e.g., 202b) includes a protrusion 214 slidably received within a groove or slot 218 of an adjacent one of the telescoping segments (e.g., 202a), to, for example, prevent rotation of the one of the telescoping segments relative to the adjacent one of the telescoping segments during operation of threaded shaft 206 and driving member or nut 210. Such a driving member or nut (e.g., 210)—or mechanism(s) of other embodiments that accomplish similar functionality—may be actuated by one or more actuators 274b (described in more detail below) and/or by physical interaction between a user and a device (e.g., 14a, 14b, and/or the like) (e.g., via a user-operable adjustment member, such as, for example, a knob, slider, and/or the like, which may be coupled to the device).

In this embodiment, body 18 includes an interior passageway 226 extending from within the body and/or through proximal end 22 and through distal end 26. For example, in the depicted embodiment, interior passageway 226 extends through pivotal connection 170 (e.g., through ball 174 and socket 178) and telescoping segments 202a and 202b (e.g., through threaded shaft 206). As will be described in more detail below, in the embodiment shown, interior passageway 226 may be configured to provide for fluid and/or electrical communication from within body 18 and/or through proximal end 22 and through distal end 26 (e.g., to facilitate chemotherapy, cryoablation, and/or electrocauterization).

In the depicted embodiment, device 14a includes a trocar 238 configured to be coupled to body 18 (e.g., disposed within interior passageway 226 of the body, in the embodiment shown) such that the trocar extends from distal end 26. For example, in this embodiment, trocar 238 is configured to extend from distal end 26 of body 18 by a distance 242 that is greater than any one of, or between any two of: 1.0, 1.5, 2.0, 2.5, 3.0, 3.5, 4.0, 4.5, 5.0. 5.5, 6.0, 6.5, 7.0, 8.0, 9.0, or 10.0 mm (e.g., between approximately 3 mm and approximately 5 mm) In the depicted embodiment, trocar 238 is slidably disposed within interior passageway 226 such that the trocar may be moved relative to body 18 between a retracted position (e.g., FIG. 4B) and a deployed position (FIG. 4A), in which less of the trocar is disposed within the body. Such slidable displacement of trocar 238 relative to body 18 may be accomplished in any suitable fashion, such as, for example, via one or more actuators 274c, described in more detail below. In the embodiment shown, trocar 238 includes a lumen in fluid communication with interior passageway 226 of body 18. In this embodiment, the lumen of trocar 238 has an internal diameter of approximately 0.1 mm; however, other embodiments may include a device (e.g., 14a, 14b, and/or the like) having a trocar (e.g., 238) with any suitable dimensions, such as, for example, having a lumen with an internal diameter that is greater than any one of or between any two of: 0.1, 0.2, 0.3, 0.4, 0.5, 0.6, 0.7, 0.8, 0.9, 1, 1.1, 1.2, 1.3, 1.4, 1.5, or 2 mm.

In the depicted embodiment, trocar 238 is configured to convey cryoablative fluid (e.g., liquid nitrous oxide, liquid nitrogen, and/or any other suitable cryoablative fluid) from a cryoablative fluid source (e.g., 254) and through its lumen. A cryoablative fluid source (e.g., 254) may be external to a device (e.g., 14a, 14b, and/or the like) and may be in communication with an interior passageway (e.g., 226) of the device via a connector (e.g., 258) or may be internal to the device (e.g., disposed within body 18). In these and similar embodiments, a trocar (e.g., 238) may be coated (e.g., inside and/or outside) with an insulative material, such as, for example, a rubber, a plastic, and/or the like. In some embodiments, a device (e.g., 14a, 14b, and/or the like) may include a trocar (e.g., 238) configured to be in electrical communication with an electrical power source (e.g., 262) via, for example, wiring or other electrical conductors disposed within an interior passageway (e.g., 226) and/or an interior channel (e.g., 70) of a body (e.g., 18) of the device, to, for example, provide for electrocauterization using the trocar. Trocars (e.g., 238) may comprise a biocompatible material, such as, for example, any one or more of those described above, and may be biodegradable.

In the embodiment shown, trocar 238 is configured to extend between magnet 42 and magnetometer 58 of at least one detector 38 (e.g., to provide for chemotherapy, cryoablation, electrocauterization, and/or the like at or proximate to abnormal tissue detected by the detector). In this embodiment, trocar 238 comprises a substantially non-ferromagnetic material, such as, for example, any one or more of those described above, to for example, mitigate interference of the trocar with a magnetometer 58 of a detector and/or a magnetic field generated by a magnet 42 of the detector.

As mentioned above, in the embodiment shown, device 14a comprises one or more actuators. For example, in this embodiment, device 14a comprises one or more actuators 274a configured to actuate pivotal connection 170 to angularly displace distal end 26 relative to proximal end 22 (e.g., via actuation of one or more rods or wires 182), one or more actuators 274b configured to actuate telescoping segments 202a and 202b to adjust a length of body 18 (e.g., via actuation of driving member or nut 210), and one or more actuators 274c configured to displace trocar 238 relative to the body. Such actuator(s) (e.g., 274a, 274b, 274c, and/or the like) may comprise any suitable actuator, such as, for example, a magnetic, piezoelectric, and/or the like actuator. Electrical power to such actuator(s) (e.g., 274a, 274b, 274c, and/or the like) may be provided by a power source, such as a battery coupled to or disposed within a device (e.g., 14a, 14b, and/or the like) and/or by a power source that is external to, but is in electrical communication with, the device.

In this embodiment, device 14a comprises a receiver 286 in communication with at least one of one or more actuators (e.g., 274a, 274b, 274c, and/or the like) and configured to receive (e.g., from a controller 310, described in more detail below) one or more commands indicative of at least one of: (1) a desired angular position of distal end 26 of body 18 relative to proximal end 22 of the body; (2) a desired length of the body; and (3) a desired position of trocar 238 relative to the body, and communicate the one or more commands to the corresponding actuator(s). In the depicted embodiment, receiver 286 may (e.g., also) be configured to receive one or more commands indicative of a desired intensity of a magnetic field to be produced by a magnet (e.g., 42) and communicate the one or more commands to the magnet, a battery in electrical communication with the magnet, and/or a controller coupled to the magnet and/or the battery. In the embodiment shown, receiver 286 is configured to communicate over a wireless connection and may communicate using any suitable communications protocol, such as, for example, Wi-Fi, Bluetooth, a cellular communications protocol, a magnetic communications protocol, and/or the like. However, other embodiments may comprise a receiver (e.g., 286) configured to communicate over a wired connection.

In this embodiment, apparatus 10 comprises one or more processors 290 configured to detect a presence of abnormal tissue based, at least in part, on data captured by magnetometer 58 of at least one of one or more detectors 38. For example, in the depicted embodiment, one or more processors 290 are configured to detect a presence of abnormal tissue at least by comparing data captured by magnetometer 58 of at least one of one or more detectors 38 to a baseline magnetic flux density. Such a baseline magnetic flux density may be a pre-determined magnetic flux density that is indicative of the presence of normal tissue or abnormal tissue, may be determined based on data captured by magnetometer 58 in the presence of (e.g., known) normal tissue or abnormal tissue, and/or the like.

Processor(s) (e.g., 290) may be coupled to and/or disposed within a device (e.g., 14a, 14b, 14c, and/or the like) and/or may be remote from, yet in communication with, the device. For example, in the embodiment shown, device 14a comprises a transmitter 294 in communication with at least one of one or more detectors 38 and configured to transmit data captured by magnetometer 58 of the detector to one or more processors 290. In this embodiment, transmitter 294 may be (e.g., further) configured to transmit data captured by one or more proximity or contact sensors 122 to one or more processors 290 and/or controller 310. In the depicted embodiment, transmitter 294 is configured to communicate over a wireless connection and may communicate using any suitable communications protocol, such as, for example, any one or more of those described above with respect to receiver 286; however, other embodiments may comprise a transmitter (e.g., 294) configured to communicate over a wired connection.

In the depicted embodiment, apparatus 10 includes a controller 310 configured to communicate with receiver 286 and/or transmitter 294. For example, in the embodiment shown, controller 310 may allow a user to send information, such as, for example, one or more commands, to receiver 286 and/or receive information, such as, for example, data captured by a magnetometer 58, a proximity or contact sensor 122, and/or the like, from transmitter 294. Controllers (e.g., 310) of the present apparatuses (e.g., 10) may or may not be hand-held.

Data captured by a magnetometer 58 of a detector 38 may be stored (e.g., in a memory) and/or displayed. For example, in the embodiment shown, apparatus 10 comprises a display 306 configured to be coupled to one or more processors 290 and to display an image indicative of data captured by a magnetometer 58 of a detector 38. In some embodiments, a display (e.g., 306) may be configured to display such an image in a color-coded format; for example, with colors representing levels of magnetic flux density. Such displays (e.g., 306) may or may not be comprised by a controller (e.g., 310).

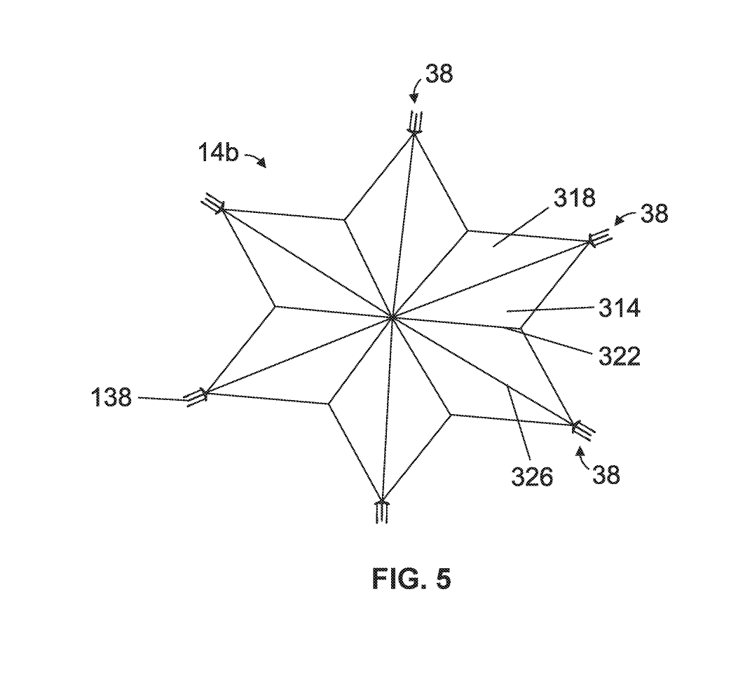

Referring now to FIG. 5, shown is a front view of a device 14b, which may be suitable for use in some embodiments of the present apparatuses (e.g., 10). Device 14b may be substantially similar to device 14a, with the primary exceptions described below. In the embodiment shown, device 14b includes a plurality of detectors 38 (e.g., six (6) detectors 38, as shown), each coupled to the periphery of a deployable scaffold 314. In this embodiment, scaffold 314 may comprise a flexible material 318 (e.g., any one or more of the flexible materials described above for flange 130) that may be supported by one or more ribs 322. In the depicted embodiment, scaffold 314 is generally star-shaped; however, other embodiments may include a scaffold (e.g., 314) having any suitable shape, such as, for example, circular, elliptical, and/or otherwise rounded, triangular, square, and/or otherwise polygonal.

Similarly to as described above for flange 130 of device 14a, in the embodiment shown, scaffold 314 is retractable or collapsible (e.g., within lumen 114 of delivery device 110). In this embodiment, scaffold 134 may be deployed (e.g., by removing device 14b from lumen 114 of delivery device 110) into a cavity at a target site within a patient such that at least a portion of the periphery of the scaffold contacts an edge or boundary of the cavity (e.g., thereby positioning detector(s) 38 coupled to the scaffold at location(s) suitable for the detection of abnormal tissue along the edge or boundary of the cavity). In some instances, a device 14a (or a similar device) may be used to create the cavity, the device may be removed, and device 14b may be inserted into the cavity. Some embodiments of the present devices, such as device 14b, may be configured to be left within a patient (e.g., to provide for real-time monitoring for and/or imaging of abnormal tissue).

In the embodiment shown, device 14b includes a trocar 138 disposed between magnet 42 and magnetometer 58 of each detector 38. In this embodiment, each trocar 138 may be configured for electrocautery (e.g., as described above). For example, in the depicted embodiment, device 14b, for each trocar 138, includes one or more wires 326 coupled to the trocar and configured to supply electrical power to the trocar for performing electrocautery. Such wires (e.g., 326) may function as and/or may comprise ribs 322 for providing support to flexible material 318. Device 14b may be configured to perform electrocautery with trocars 138 individually and/or collectively.

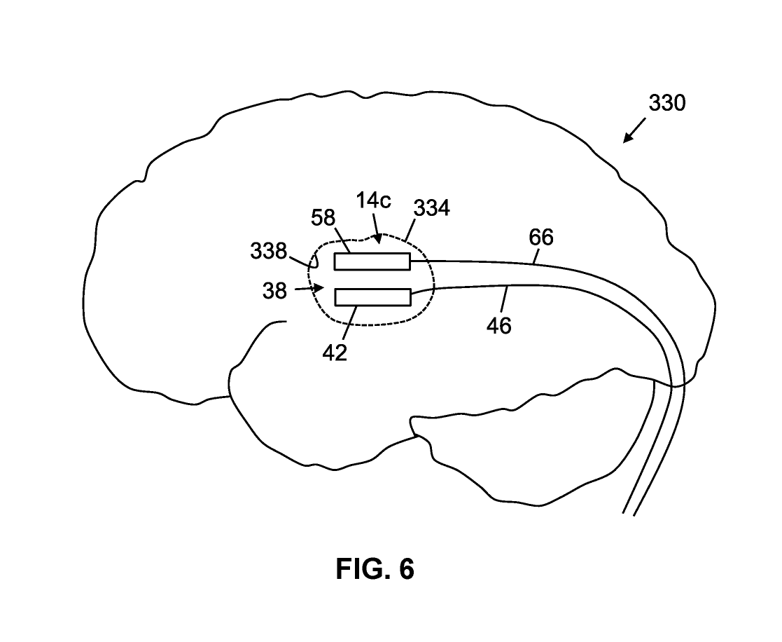

Referring now to FIG. 6, shown is a side view of a device 14c, which may be suitable for use in some embodiments of the present apparatuses (e.g., 10). Device 14c may be substantially similar to device 14a, with the primary exceptions described below. In this embodiment, device 14c comprises a flexible, wirelike coupling between detector 38 and other component(s) of the device and/or an apparatus (e.g., 10) comprising the device (e.g., a power source, processor 290, and/or the like). In the depicted embodiment, via the flexible, wirelike coupling, detector 38 can be disposed within one area of a patient's body (e.g., brain 330), and other component(s) of device 14c and/or an apparatus (e.g., 10) comprising the device can be disposed on and/or within another area of the patient's body (e.g., the patient's skin, neck, torso, and/or the like). For example, in the embodiment shown, device 14c comprises one or more flexible wires (e.g., 46 and 66). In this embodiment, at least one of the flexible wire(s) (e.g., 66) is configured to be in electrical communication with magnetometer 58. In the depicted embodiment, at least one of the flexible wire(s) (e.g., 46) is configured to be in electrical communication with magnet 42. In some embodiments, such flexible wire(s) (e.g., 46 and/or 66) can be disposed within a flexible sheath.

Some embodiments of the present methods comprise detecting a presence of abnormal tissue within a patient using any apparatus (e.g., 10) and/or device (e.g., 14a, 14b, 14c) of the present disclosure. For example, some embodiments of the present methods comprise inserting a detector (e.g., 38) of a device (e.g., 14a, 14b, 14c, and/or the like) into a target site on a patient (e.g., which may be located using CT tomography, MRI, or any other suitable imaging technique), the detector including a magnet (e.g., 42) and a magnetometer (e.g., 58). In some embodiments, the inserting comprises inserting an elongated delivery device (e.g., 110) into the target site, the detector being disposed within a lumen (e.g., 114) of the delivery device, removing the detector from the lumen of the delivery device, and removing the delivery device from the target site. In embodiments where the target site is beneath bone, such as, for example, where the target site is within the patient's brain, a hole (e.g., a burr hole) may be made through the bone to facilitate insertion of the device.

In some embodiments, data captured by an oximeter or other suitable sensor (e.g., which may be coupled to the device or the delivery device) may be used to reduce the risk of puncturing or otherwise damaging blood vessels during insertion of the device. For example, in some embodiments, if data captured by an oximeter or other suitable sensor indicates an oxygen saturation in an area that is greater than 95%, that area may be avoided (e.g., by redirecting the device or delivery device) to avoid puncturing or otherwise damaging blood vessel(s) in the area. One example of a suitable oximeter is disclosed in U.S. Pub. No. 2015/0073240, which is hereby incorporated by reference in its entirety.

In some embodiments, the inserting comprises forming a cavity at or proximate to the target site and inserting the detector into the cavity. In some embodiments, forming the cavity comprises cryoablation (e.g., passing cryoablative fluid from cryoablative fluid source 254 through trocar 238). In some embodiments, forming the cavity comprises electrocauterization (e.g., passing electrical power from electrical power source 262 through trocar 238). In these and similar embodiments, at least by forming such a cavity, room for insertion and/or actuation of the device (e.g., angular displacement of distal end 26 relative to proximal end 22, adjustment of a length of body 18, displacement of trocar 238 relative to the body, and/or the like) may be provided.

Some embodiment comprise producing a magnetic field with the magnet, moving the detector relative to the patient, and capturing, with the magnetometer, data indicative of disturbances in the magnetic field. In some embodiments, the moving comprises moving the detector along a boundary of the cavity (e.g., which may be facilitated by one or more proximity or contact sensors 122). In some embodiments, such movement may be facilitated by a pivotal connection (e.g., 170), telescoping segments (e.g., 202a, 202b, and/or the like), and/or the like.

Some embodiment comprise analyzing data captured by the magnetometer to detect a presence of abnormal tissue at or proximate to the target site. For example, in some embodiments, data captured by the magnetometer includes data indicative of a magnetic flux density. In some embodiments, the analyzing comprises comparing data captured by the magnetometer to a baseline magnetic flux density. Such a baseline magnetic flux density may be a pre-determined magnetic flux density that is indicative of the presence of normal tissue or abnormal tissue, may be determined based on data captured by the magnetometer in the presence of (e.g., known) normal tissue or abnormal tissue, and/or the like.

Some embodiments comprise ablating tissue at or proximate to the target site. In some embodiments, the ablating comprises cryoablation (e.g., passing cryoablative fluid from cryoablative fluid source 254 through trocar 238). In some embodiments, the ablating comprises electrocauterization (e.g., passing electrical power from electrical power source 262 through trocar 238). In some embodiments, such ablating may continue until data captured by the magnetometer no longer indicates a presence of abnormal tissue at or proximate to the target site.

Some embodiments of the present methods comprise inserting a detector (e.g., 38) of a device (e.g., 14a, 14b, 14c, and/or the like) into a target site of a patient, the detector including a magnet (e.g., 42) and a magnetometer (e.g., 58), producing a magnetic field with the magnet, and capturing, with the magnetometer, data indicative of a magnetic flux density and/or disturbances in the magnetic field. Some embodiments comprise analyzing data captured by the magnetometer to detect a presence of abnormal tissue at or proximate to the target site. In some embodiments, the analyzing data captured by the magnetometer comprises comparing data captured by the magnetometer to a baseline magnetic flux density.

In some embodiments, the capturing data with the magnetometer is performed while producing the magnetic field with the magnet, and, optionally, the magnetic field produced by the magnet is constant. Some embodiments comprise increasing an intensity of the magnetic field produced by the magnet in response to data captured by the magnetometer.

In some embodiments, the device comprises one or more flexible wires (e.g., 46 and/or 66), at least one of the one or more flexible wires (e.g., 66) is configured to be in electrical communication with the magnetometer, and, optionally, at least one of the one or more flexible wires (e.g., 46) is configured to be in electrical communication with the magnet. In some embodiments, the device includes an elongated body (e.g., 18) extending between a proximal end (e.g., 22) and a distal end (e.g., 26), and the detector is coupled to the distal end of the elongated body.

In some embodiments, the inserting the detector comprises inserting an elongated delivery device (e.g., 110) into the target site, the detector being disposed within a lumen of the delivery device, removing the detector from the lumen of the delivery device, and removing the delivery device from the target site.

In some embodiments, the target site is within the patient's brain (e.g., 330). In some embodiments, the target site of the patient comprises a cavity (e.g., 334) that was previously occupied by abnormal tissue. Some embodiments comprise moving the detector along a boundary (e.g., 338) of the cavity.

In some embodiments, the patient has been previously diagnosed as having cancer, and/or the patient has been determined to have a genetic predisposition to cancer.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An apparatus for in vivo detection of abnormal tissue, the apparatus comprising:
a device including a detector configured to be inserted into a patient, the detector having:
a magnet configured to produce a magnetic field; and
a magnetometer spaced apart from the magnet and configured to capture data indicative of a magnetic flux density; and
a processor configured to analyze data captured by the magnetometer to detect a presence of abnormal tissue within the patient.

2. The apparatus of claim 1, wherein:
the device comprises one or more flexible wires;
at least one of the one or more flexible wires is configured to be in electrical communication with the magnetometer; and
optionally, at least one of the one or more flexible wires is configured to be in electrical communication with the magnet.

3. The apparatus of claim 1, wherein:
the device includes an elongated body extending between a proximal end and a distal end; and
the detector is coupled to the distal end of the elongated body.

4. The apparatus of claim 3, wherein the detector is movable between a deployed state and a retracted state in which the magnet is closer to the magnetometer than when the detector is in the deployed state.

5. The apparatus of claim 4, wherein:
the distal end of the elongated body defines a flange such that at least a portion of the flange is proximal to the detector; and
the flange is movable between:
a collapsed state in which the flange has a first maximum transverse dimension; and
a deployed state in which the flange has a second maximum transverse dimension that is larger than the first maximum transverse dimension.

6. The apparatus of claim 5, wherein movement of the flange toward the deployed state causes movement of the detector toward the deployed state.

7. The apparatus of claim 1, wherein the detector is disposable through a lumen of an elongated delivery device.

8. The apparatus of any of claims 1-7, wherein the processor is configured to analyze data captured by the magnetometer at least by comparing data captured by the magnetometer to a baseline magnetic flux density.

9. A method comprising:
inserting a detector of a device into a target site of a patient, the detector including:
a magnet; and
a magnetometer;
producing a magnetic field with the magnet; and
capturing, with the magnetometer, data indicative of:
a magnetic flux density; and/or
disturbances in the magnetic field.

10. The method of claim 9, wherein:
the device comprises one or more flexible wires;
at least one of the one or more flexible wires is configured to be in electrical communication with the magnetometer; and
optionally, at least one of the one or more flexible wires is configured to be in electrical communication with the magnet.

11. The method of claim 9, wherein:
the device includes an elongated body extending between a proximal end and a distal end; and
the detector is coupled to the distal end of the elongated body.

12. The method of claim 9, wherein:
the capturing data with the magnetometer is performed while producing the magnetic field with the magnet; and
optionally, the magnetic field produced by the magnet is constant.

13. The method of claim 9, comprising increasing an intensity of the magnetic field produced by the magnet in response to data captured by the magnetometer.

14. The method of claim 9, comprising analyzing data captured by the magnetometer to detect a presence of abnormal tissue at or proximate to the target site.

15. The method of claim 14, wherein the analyzing data captured by the magnetometer comprises comparing data captured by the magnetometer to a baseline magnetic flux density.

16. The method of claim 9, wherein the target site of the patient comprises a cavity that was previously occupied by abnormal tissue.

17. The method of claim 16, comprising moving the detector along a boundary of the cavity.

18. The method of claim 9, wherein the target site is within the patient's brain.

19. The method of claim 9, wherein the inserting the detector comprises:
inserting an elongated delivery device into the target site, the detector being disposed within a lumen of the delivery device;
removing the detector from the lumen of the delivery device; and
removing the delivery device from the target site.

20. The method of any of claims 9-19, wherein:
the patient has been previously diagnosed as having cancer; and/or
the patient has been determined to have a genetic predisposition to cancer.
